(19) United States
(12) Patent Application Publication
(10) Pub. No.: US 2023/0366151 A1
(43) Pub. Date: Nov. 16, 2023

(54) NITROCELLULOSE PAPERS AND METHODS OF PRODUCTION AND USE

(71) Applicant: MERCK PATENT GMBH, DARMSTADT (DE)

(72) Inventors: Tina BOCK, Darmstadt (DE); Markus BIESALSKI, Darmstadt (DE); Marcel KRAUSSE, Darmstadt (DE); Tobias MECKEL, Darmstadt (DE); Dieter SPIEHL, Darmstadt (DE)

(73) Assignee: MERCK PATENT GMBH, DARMSTADT (DE)

(21) Appl. No.: 18/029,220

(22) PCT Filed: Sep. 28, 2021
(86) PCT No.: PCT/EP2021/076646

§ 371 Date: Mar. 29, 2023

(30) Foreign Application Priority Data
Sep. 30, 2020 (EP) 20199154.4

(51) Int. Cl. D21H 19/34; D21H 21/24; D21H 23/46; D21H 23/34; D21H 23/56; D21H 23/50; G01N 33/543; B01D 71/20; B01D 69/10; B01D 69/02; B01D 67/00

(52) U.S. Cl. CPC D21H 19/34; D21H 21/24; D21H 23/46; D21H 23/34; D21H 23/56; D21H 23/50; G01N 33/54386; B01D 71/20; B01D 69/107; B01D 69/02; B01D 67/0009; B01D 67/0095; B01D 2325/0283; B01D 2325/04

(57) ABSTRACT

The present invention relates to cellulose based papers coated with nitrocellulose as well as to methods for producing such coated papers and methods for using them, especially in lateral flow applications.

The present invention relates to cellulose based papers coated/impregnated with nitrocellulose as well as to methods for producing such coated papers and methods for using them, especially in lateral flow applications.

Several analytical assay formats are based on the use of porous materials like membranes which enable the flow of liquid through the porous membrane and allow for immobilization of molecules. Nitrocellulose membranes have been used in traditional protein blotting methods, high throughput arrays, mass spectrometry proteomics and immunodiagnostics. One example is lateral flow tests. These devices can be used to confirm the presence or absence of a target analyte, such as pathogens or biomarkers in humans or animals, or contaminants in water supplies, foodstuffs, or animal feeds. The most commonly known type of lateral flow rapid test strip is the pregnancy test.

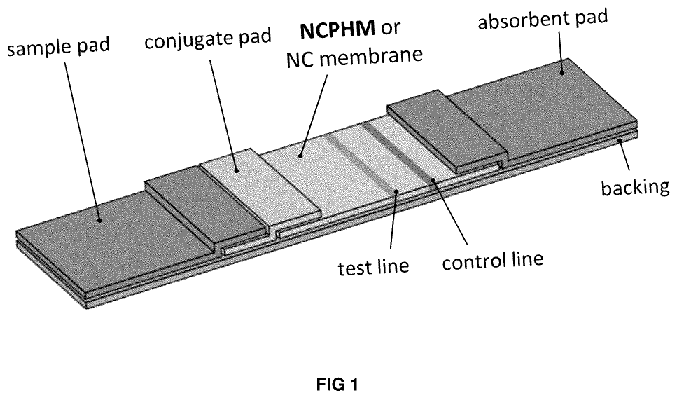

The principle of a lateral flow test is known to a person skilled in the art: a liquid sample (or its extract) containing the analyte of interest moves without the assistance of external forces (by capillary action) through typically various zones of porous membrane strips, on which molecules that can interact with the analyte are attached. A typical lateral flow test strip consists of overlapping porous materials that are mounted on a backing card for better stability and handling. The sample is applied at one end of the strip, the start zone, which is an absorbent sample pad typically impregnated with buffer salts and surfactants that make the sample suitable for interaction with the detection system. The sample pad ensures that the analyte present in the sample will be capable of binding to the capture reagents on conjugates and on the membrane. The treated sample migrates through the conjugate release pad, which contains antibodies that are specific to the target analyte and are conjugated to coloured or fluorescent particles—most commonly colloidal gold and latex microspheres. The sample, together with the conjugated antibody bound to the target analyte, migrates along the strip into the detection zone. This is a porous membrane usually composed of nitrocellulose with specific biological components (mostly antibodies or antigens) immobilized in lines or other defined formats. Their role is to react with the analyte bound to the conjugated antibody. Recognition of the sample analyte results in an appropriate response on the test line, while a response on the control line indicates the proper liquid flow through the strip. The read-out, represented by the lines appearing with different intensities, can be assessed by eye or using a dedicated reader. In order to test multiple analytes simultaneously under the same conditions, additional test lines of antibodies specific to different analytes can be immobilized in an array format. An exemplary lateral flow device is shown in FIG. 1.

The membrane used for the detection zone is typically considered the most critical element in a lateral flow test as it needs to fulfil several requirements. First, the specific biological components, typically antibodies or antigens need to be stably immobilized on the membrane so that they do not leak or move away and thus hinder accurate detection. Consequently, the membrane needs to allow for effective binding and immobilizing of e.g. proteins necessary for subsequent selection, reaction and detection. In addition, the membrane needs to allow for homogenous capillary flow so that the liquid comprising the labeled analyte can easily and homogenously access the immobilized proteins. In addition, the flow needs to be efficient enough to distribute the liquid in a certain time frame. Nitrocellulose membranes are by far the most commonly used material for the detection zone. Such membranes are well known and established and consist of a polymeric foil made of e.g. PET or polypropylene as a substrate which is coated with a thin and porous nitrocellulose layer. These membranes have a high internal surface area due to the porous structure and are able to bind ample amounts of proteins via electrostatic/hydrophobic interactions.

However, despite their widespread use, these membranes do have some drawbacks. Nitrocellulose is a relatively expensive material, flammable, and brittle and thus needs, in most cases, lamination to a backing foil to prevent destruction during handling. Furthermore, as protein adsorption is nonspecific, NC membranes are generally required to be blocked (e.g. with BSA) prior to the analysis of protein containing samples.

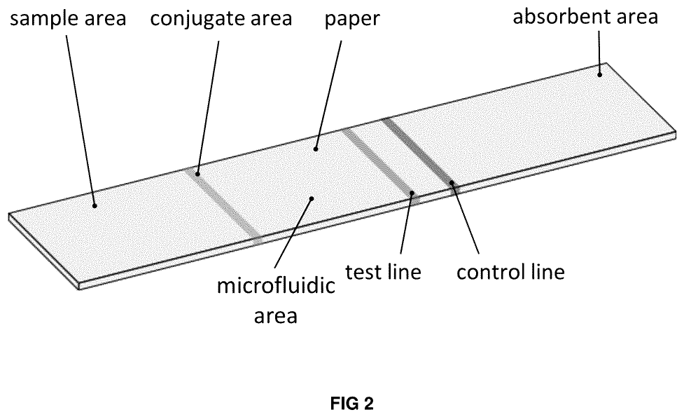

It has been found by the present inventors that nitrocellulose hybrid materials can be generated by coating cellulose based papers with a nitrocellulose lacquer. The resulting nitrocellulose papers show all good properties concerning stability, immobilization of proteins and porosity. They can advantageously be used in immunoassays like lateral flow assays. It is even possible to generate a “one piece” lateral flow device in which the partially nitrocellulose coated paper is used as sample pad, conjugate release pad, detection zone etc. without the need to combine different materials. A schematic view of a one-piece lateral flow device is shown in FIG. 2. It has been found that using a lacquer with a certain composition provides nitrocellulose papers which can be suitably also used for other applications like protein depletion or delay, filtering or Western Blot.

The present invention is thus directed to a nitrocellulose coated paper comprising a cellulose paper sheet partially or fully coated with nitrocellulose and further treated with a detergent after the nitrocellulose coating. The detergent treatment can also be partially or fully. ‘Partially’ preferably means spatially resolved, whereby the coating or treatment is only applied to certain, defined areas of the paper sheet.

In a preferred embodiment the nitrocellulose coated paper is preparable by contacting a cellulose paper sheet with a lacquer comprising nitrocellulose, a detergent, at least one solvent in which nitrocellulose is soluble and at least one non solvent pore former which is a solvent in which nitrocellulose is insoluble.

In a preferred embodiment the nitrocellulose coated paper has a porosity between 50 and 80% (v/v).

In another preferred embodiment the cellulose paper sheet has pores with sizes between 3 μm and 30 μm.

In another preferred embodiment the nitrocellulose coating has pores between 0.2 μm and 15 μm.

In another embodiment the nitrocellulose paper comprises at the fully coated parts between 1% and 40% nitrocellulose (w/w).

In a preferred embodiment, the lacquer comprises 1 to 20% (w/w) of nitrocellulose.

In another preferred embodiment the lacquer comprises 0.05 to 2% (w/w) of a detergent, preferably 0.05 to 1%. Preferably the detergent is an alkylphenol ethoxylate.

In a preferred embodiment the nitrocellulose coated paper is further treated with an anionic detergent.

The present invention is further directed to a method for preparing nitrocellulose papers by
a) Providing a cellulose paper sheet and a lacquer comprising nitrocellulose, at least one solvent in which nitrocellulose is soluble and at least one non solvent pore former
b) Contacting the cellulose paper sheet with the lacquer
c) Drying the coated paper
d) Treating the dried coated paper with a detergent

In a preferred embodiment in step b) the cellulose based paper is contacted with the lacquer by dip coating, blade coating, size press coating or printing.

The present invention is further directed to a lateral flow device comprising a nitrocellulose coated paper.

The present invention is further directed to the use of the nitrocellulose coated papers for lateral flow tests, filtration, as protein barrier, for delaying flow or for western blot assays.

FIG. 1 shows a schematic view of a lateral flow device.

FIG. 2 shows a schematic view of a one-piece lateral flow device.

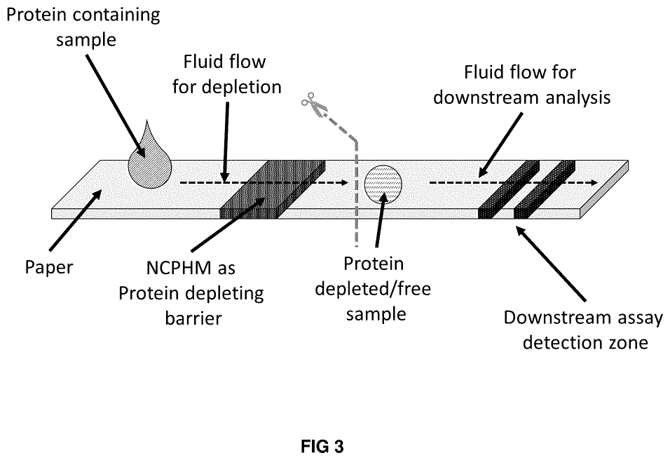

FIG. 3 shows a schematic view of a device for protein depletion. Further details can be found in Example 6.

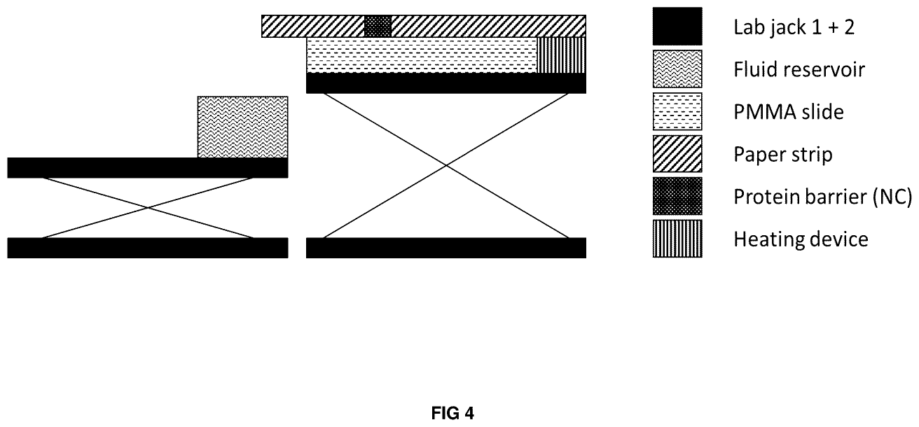

FIG. 4 shows the set up for testing protein depletion. Further details can be found in Example 6.

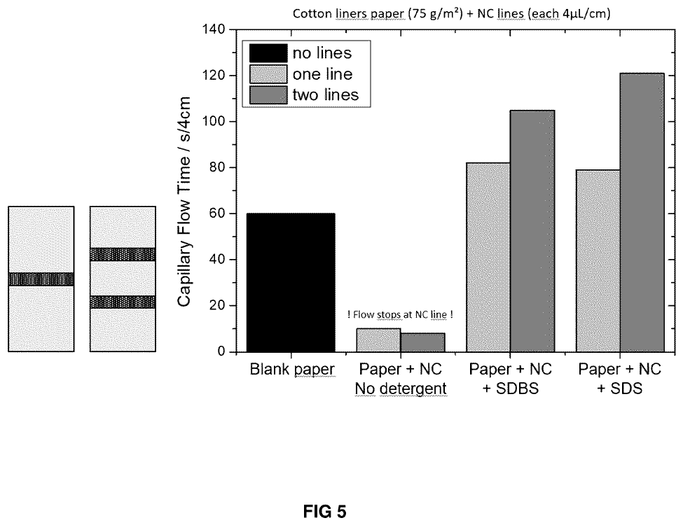

FIG. 5 shows the scheme of prepared hybrids and capillary flow times of hybrids without detergent and detergents. Further details can be found in Example 7.

As used herein, the term “detergent” refers to molecules having lipophilic as well as hydrophilic (i.e. amphiphilic) characteristics. A detergent according to the present invention may be an anionic, a cationic, a zwitterionic or a non-ionic detergent. Preferably, it is an ionic detergent. Suitable detergents comprise, for instance, a fatty acid residue and a hydrophilic (e.g. anionic or cationic) part. Examples of suitable detergents are sorbitan alkyl esters, polyethylene glycol sorbitan alkyl esters, polyethylene glycol alkyl ethers, glycerol alkyl esters, glucoside alkyl ethers and alkyl polyglycosides, block copolymers of polyethylene glycol and polypropylene glycol as well as alkylphenol ethoxylates like e.g. octylphenol ethoxylate, nonylphenol ethoxylate and tributyl phenol ethoxylate. Exemplary detergents are sodium dodecylsulfate (SDS), CHAPS, Lutensol® AO-7, SDBS, Tween®80, Triton®X100.

Pore size is determined by Hg porosimetry. Pore size distributions for the pores of the papers of the present invention were obtained using Hg porosimetry, which technique is described by Winslow and Shapiri in “An Instrument for Measurement of Pore Size Distribution by Mercury Penetration,” ASTM Bulletin, February 1959. Mercury contact angle in the pore is particularly important in pore size distribution calculations based on mercury penetration data. A contact angle of 140 DEG was assumed in calculating all the pore size distributions referred to herein.

The pore size according to the present invention is a pore size distribution which means that the size of more than 70%, preferably more than 80%, most preferred more than 90% of the pores is of the given size or within the given size range.

Nitrocellulose (also known as cellulose nitrate, flash paper, flash cotton, guncotton, and flash string) is a highly flammable compound formed by nitrating cellulose through exposure to a mixture of concentrated nitric acid and sulfuric acid or another powerful nitrating agent. Exemplary suitable nitrocelluloses are nitrocellulose materials having a nitrogen content between 10 and 12% (w/w) and a molecular weight range between 1.0·10³ and 1.0·10⁶. Blending of different nitrocellulose materials with e.g. different molecular weight ranges is also possible.

Nitrocellulose is manufactured as an industrial commodity. The polymer is typically characterized on the basis of solution viscosity at defined concentrations and related back to mean molecular weight. While this measurement indicates the bulk properties of a given batch of nitrocellulose, blending different grades of polymers to produce a desired viscosity leads to variation in the molecular weight distribution between lots.

This variation affects both the dissolution properties and the precipitation characteristics.

Further details can be found in R. C. Wong, H. Y. Tse (eds.), Lateral Flow Immunoassay, DOI 10.1007/978-1-59745-240-3_6, Chapter 6.

For the solvent component, all organic solvents in which nitrocellulose is soluble are suitable. In this context, ‘soluble’ means that the amount of nitrocellulose used for the production of the lacquer dissolves in the amount of the solvent component at room temperature within about 2 hours of stirring. Suitable solvents may vary depending on the type of nitrocellulose that is used. Nitrocellulose with a shorter chain length is typically soluble in other solvents that nitrocellulose with longer chain length is not. Examples are acetone, tetrahydrofuran, ethyl acetate, isopropanol, propanol, butanol and methylene chloride, whereby acetone and tetrahydrofuran are especially suitable for nitrocellulose with longer chain length.

The nonsolvent pore formers are solvents in which nitrocellulose is insoluble under the conditions described above but which are miscible with the nitrocellulose dissolving solvents. The preferred nonsolvent pore former is water.

The present invention is directed to a nitrocellulose coated paper whereby the nitrocellulose coated paper, also called Nitrocellulose-Paper Hybrid material (NCPHM), consists of a base sheet of cellulose paper which is coated and thus impregnated with a nitrocellulose (NC) solution (lacquer) followed by a drying process. The applied NC is not covalently bound to the cellulose paper. It is located on the paper surface and optionally inside the fiber network. The thickness of the NCPHM is typically between 50 μm and 500 μm, preferably between 100 μm and 300 μm.

It was found that the paper provides a stable and flexible support in combination with useful properties for fluid transport, and NC provides a higher specific surface area and protein binding properties, owing to its chemical and structural nature.

The nitrocellulose coated paper according to the present invention comprises a cellulose paper sheet which is coated with nitrocellulose and further treated with a detergent.

Suitable cellulose papers have a content of more than 95% (w/w) of cellulose. Preferably they consist of pure cotton linters fibers or eucalypt fibers. The papers preferably have grammages from 30 g/m²-250 g/m², preferably 50 g/m²-155 g/m². The porosity of the paper is preferably between 50-80%. Typical paper thickness is between 100 and 300 μm. Exemplary fiber length of cotton linters papers is between 0.45 mm-1.15 mm, preferably between 0.6 mm-0.9 mm and a fiber diameter from 16 μm-22 μm. Further exemplary suitable paper properties are
Fiber fibrillation: 1.65%-2.31%
Schopper-Riegler Value (drainability): 17°-65°
Crude density: 0.42 g/m³-0.57 g/m³
Specific volume: 1.75 cm³/g-2.40 cm³/g

Suitable cellulose papers are commercially available. They can also be produced according to the procedure disclosed in Example 1.

The coating of the cellulose paper sheet is done by contacting it with a lacquer. The lacquer is a liquid comprising at least nitrocellulose, a solvent in which nitrocellulose is soluble, a nonsolvent pore former and optionally a detergent. Depending on the properties needed and the final use, it might also comprise other additives. The nitrocellulose is typically present in the lacquer in an amount between 1% and 20% (w/w), preferably between 2 and 15% (w/w). One has to take into account that the weight % of nitrocellulose includes wet alcohol, typically around 30% (w/w), as the suitable raw material is nitrocellulose with alcohol.

A preferred solvent is acetone. It is typically present in the lacquer in an amount between 25 and 60% (w/w), preferably between 30 and 45% (w/w).

Suitable nonsolvent pore formers are water or, in case of nitrocellulose with a longer chain length, C1 to C4 alcohols like ethanol, iso-propanol, iso-butanol or n-butanol, as well as mixtures thereof.

Typically, the lacquer comprises between 20 and 70% (w/w), preferably between 40 and 60% (w/w) of the nonsolvent pore former. Preferably, the nonsolvent pore former is a mixture of two or three solvents selected from water, ethanol, iso-propanol and n-butanol.

The lacquer preferably also comprises a detergent. The detergent can be any detergent as described above. One suitable detergent is Triton®X-100. The detergent is typically present in an amount between 0.05 and 1 (w/w), preferably between 0.1 and 0.3% (w/w).

The lacquer may also comprise further additives like cellulose esters or glycerol, e.g. to adjust the properties of the nitrocellulose coating to the application for which it shall be used. Exemplary cellulose esters are cellulose acetate, cellulose acetate phthalate (CAP) and cellulose acetate butyrate (CAB). Suitable concentrations of cellulose esters, if present, are between 0.05 and 1.5% (w/w). Suitable concentrations of glycerol, if present, are between 0.3 and 2% (w/w). The table shown in Example 2 provides further exemplary ranges for suitable lacquers. An exemplary lacquer composition is 10% nitrocellulose, 27% acetone, 10% ethanol, 45% iso-propanol, 0.1% Triton®X100, 7.9% water (all w/w). A person skilled in the art can adjust the composition of the lacquer to the respective purpose of the nitrocellulose coating or impregnation.

It has been found that by contacting the paper with a lacquer as defined above, the nitrocellulose coating can be applied homogenously, stable and with a homogenous pore structure. Depending on the concentration of the nitrocellulose in the lacquer, the paper fibers are coated with the nitrocellulose (in case of a less concentrated lacquer) or also the pores of the paper are partly or completely filled with a 3-dimensional network of nitrocellulose (in case of a more concentrated lacquer).

Also the thickness of NC coat on the fibers can be varied. It may be between 2-20 μm depending on the NC content and coating method. High NC contents and methods like dip coating or blade coating lead to thicker NC coat on the paper. Low NC contents in general lead to thinner NC coat on the paper.

The paper can be fully or partially coated with the nitrocellulose lacquer. Coating means that either the surface of the paper or parts of the surface of the paper can be coated with nitrocellulose or that part of the paper or the whole sheet of paper is penetrated and impregnated with the lacquer and the surface as well as the inner fibers of the paper are coated with the nitrocellulose. Optionally, the pores of the paper are fully or partially filled with a three-dimensional network of porous nitrocellulose.

The nitrocellulose coated paper of the present invention can be prepared by any means suitable to contact the paper with the lacquer. Examples of suitable methods are dip coating, blade coating, size pressing, and printing like line printing.

Dip coating is a method for a complete impregnation of the cellulose paper with nitrocellulose lacquer. The lacquer penetrates completely through the paper and leads to the formation of porous nitrocellulose in/on the paper upon drying. Depending on the nitrocellulose content of the lacquer, complete filling of macropores in the material can be obtained. In this case the nitrocellulose content is typically above 5% (w/w). To primarily coat the fibers on the surface, a lacquer with a nitrocellulose below 5% (w/w) is suitable.

For dip coating, the cellulose papers are preferably placed for 2 h in a climate controlled environment, e.g. a climate cabinet, with defined temperature and humidity to equilibrate. Suitable conditions are 23° C. and 50% relative humidity, but other conditions are also possible. Preferably, the environmental conditions are stable during production. For dip coating, the paper sheets are then dipped into the lacquer. This is preferably also done in a climate controlled environment. If a dip coater is used, the paper sheets are typically clamped on the horizontal and movable traverse of the dip-coater. Underneath the traverse, a beaker filled with lacquer is placed and the coating process is started. The traverse travels down until the paper is completely immersed in the lacquer. After resting some time, typically some seconds, the traverse travels up with the chosen coating speed. For drying, the coated papers are preferably left in a climate cabinet for a suitable time, e.g. 1 to 5 hours. Further details can be found in Example 3.

Blade coating is usually described as a method for surface coatings of low/non wicking materials resulting in thin coated surface layers. A defined volume of lacquer is placed on the substrate and spread via a blade/rod moving over the surface with a defined gap between blade/rod and paper substrate. In the case of NCPHMs, the paper substrate is a wicking material, leading to complete impregnation of the paper with the coating lacquer. The NC is located mostly at regions of high capillary forces (fiber crossings) and doesn't span the macropores. Further details can be found in Example 3.

Size press coating is a method to immerse paper sheets completely, homogeneously and from both sides with a coating solution. The paper substrate passes through an immersion bath and enters between two rubber rollers (material preferably EPDM) which are pressed together with a defined linear pressure. The rubber rollers pull the paper through the bath and squeeze out residual coating solution, resulting in low coating weights. The NC is located at the regions of higher capillary forces (e.g. fiber crossings) and is unable to span the macropores. For size press coating, the size press (SP 6513, Werner Mathis AG, Switzerland) equipped with EPDM rubber rollers can be used. Further details can be found in Example 3.

It is also possible to use methods which allow for spatially resolved application of the lacquer, e.g. printing. Any printing equipment suitable for applying liquid inks is suitable. One example for such a method is line printing. Via a syringe deposition 3D printer or any other printer, NC lacquer can be printed in thin lines onto the paper. The resulting hybrid materials comprise only a tiny amount nitrocellulose. Further details can be found in Example 3. Other suitable methods are screen printing, gravure printing, flexographic printing, inkjet printing, spray coating or segmented coating or methods like slot die coating, case knife coating, pattern coating or strip coating.

To achieve even better accessibility of the surface of the nitrocellulose paper and thus better flow of liquid though the NCPHM, after application of the lacquer and drying, the NC coated paper is further treated with at least one detergent, preferably an anionic detergent and/or a zwitterionic detergent and/or a nonionic detergent. The detergent can be added to the NCPHM by any suitable method. Typically, a detergent solution is added; and this can be done by similar methods as for applying the nitrocellulose lacquer. Most preferred is dipping in a detergent solution. The NCPHMs can for example be placed for 10 min in a tray filled with detergent solution at room temperature (19° C.-25° C.). Afterwards, the NCPHMs are taken out and dried at room temperature overnight.

The detergent solution can for example have a detergent concentration of 0.005% to 5%, preferably 0.02% to 2%. The solvent is preferably water. Other concentrations might also be suitable depending on the detergent used and the envisaged application. The additional coating with a detergent alters the wetting properties of the NCPHM and thus the flow of liquids therethrough. Porous NC coatings are owing contact angles of about 110°, which leads to a hydrophobic interaction with water. Water is unable to penetrate the porous NC due to its high surface tension. It has been found that by applying a detergent, the surface tension of water and the contact angle decrease (<90°), leading to penetration of the fluid into the porous NC coating.

The anionic detergent is preferably sodium dodecyl sulfate (SDS), sodium dodecyl benzene sulfonate (SDBS), lithium dodecyl sulfate (LDS) or deoxycholate (DOC).

The zwitterionic detergent is preferably 3-[(3-cholamidopropyl) dimethylammonio]-1-propanesulfonate (CHAPS) or 3-[(3-Cholam-idopropyl)dimethylammonio]-2-hydroxyl-propanesulfonate (CHAPSO).

The nonionic detergent is preferably an ethoxylated aliphatic alcohol, preferably comprising a C13 to C15 aliphatic alcohol. Such ethoxylated aliphatic alcohols are also known as Lutensol. Suitable nonionic detergents are, in particular, acyl-, alkyl-, oleyl- and alkylarylethoxylates. These products are obtainable, for example on the market under the name Genapol or Lutensol. This covers, for example, ethoxylated mono-, di- and trialkylphenols (EO (ethyleneoxy group) degree: 3 to 50, alkyl substituent radical: C4 to C12) and also ethoxylated fatty alcohols (EO degree: 3 to 80; alkyl radical: C8 to C36), especially C12-C14-fatty alcohol (3-8) ethoxylates, Cl3-C15-oxo alcohol (3-30) ethoxylates, C16-C18-fatty alcohol (11-80) ethoxylates, ClO-oxo alcohol (3-11) ethoxylates, C13-oxo alcohol (3-20) ethoxylates, polyoxyethylenesorbitan monooleate having 20 ethylene oxide groups, copolymers of ethylene oxide and propylene oxide having a minimum content of 10% by weight of ethylene oxide, the polyethylene oxide (4-20) ethers of oleyl alcohol and also the polyethene oxide (4-20) ethers of nonyl phenol. Use may also be made of mixtures of said nonionic detergents.

The NCPHM according to the present invention can be used for several applications, e.g. as a substitute for nitrocellulose membranes. Exemplary applications are lateral flow assays, application where a protein barrier is needed, for delay of flow, western blot applications or as filter membrane.

Lateral flow assays are known to a person skilled in the art. The general set up has been described above. The NCPHMs according to the present invention can be used preferably as membrane of the detection zone. For this, they are treated equivalent to the known nitrocellulose membranes. For example, binding and immobilizing of e.g. proteins necessary for subsequent selection, reaction and detection are adsorbed via typically electrostatic/hydrophobic interactions.

It is also possible to use the NCPHMs of the present invention in a one-piece lateral flow assay based on a single sheet. In this case the single sheet is made of a cellulose paper sheet which is only partly coated with nitrocellulose and a detergent. Preferably, the coating with nitrocellulose and a detergent is in the detection zone. The coating might be in the form of one or more areas, lines, symbols etc. The coating can for example be applied by printing a lacquer onto the cellulose paper sheet, e.g. by line printing. The lateral flow devices comprising a nitrocellulose coated cellulose paper, either in form of a membrane substitute in the detection zone, like in case of a standard lateral flow device, or in the form of one or more printed areas like in case of assembly free one-piece lateral flow assays, can be used the same way as known lateral flow assays and for the same applications.

The NCPHMs of the present invention can further be used as protein barrier for depletion applications. It has been found that based on the high protein binding affinity of nitrocellulose, even in presence of detergent, NCPHMs can be used as potential protein barrier in (micro-) fluidic devices. This barrier will be applicable in a one-step separation of proteins and other compounds in diagnostics, e.g. protein depletion from blood or urine, or as protein barrier for bio-lab samples. For this application, typically only partially coated NCPHMs are used. Preferably, cellulose paper sheets are partially coated with the respective lacquer by printing, e.g. line printing. In one embodiment, the protein barrier can be part of a lateral flow device. In this case, the lateral flow device comprises and additional area between the start zone and the detection zone which is coated with nitrocellulose according to the present invention. This can also be seen in FIG. 3. The lateral flow test can be a one-piece lateral flow assay as shown in FIG. 2 or can be assembled. In any case the sample applied to the start zone passed through a nitrocellulose coated paper sheet according to the present invention. This area might be a line, several lines or any other format. When the sample passes through this area, the proteins present in the sample are bound to the nitrocellulose. The rest of the sample which does not bind to the nitrocellulose passes through this area without being bound to it and can then enter the further zones of the lateral flow assay so that the analysis and detection can take place with a sample that does not comprise a detectable amount of potentially hindering proteins. It is also possible to remove or cut off the part of the strip for protein depletion after the sample has passed it. This is shown by the scissors in FIG. 3. Protein depletion is especially easy to make when using one-piece lateral flow devices as instead of only printing the nitrocellulose lacquer to the detection zone one simply prints an additional area after the start zone.

In some analytic and diagnostic assays, time delays of a fluid flow can be beneficial. Time delays can lead to a longer residence time on sensor fields in microfluidic devices (e.g. Lateral Flow Assays) leading to an increased sensitivity and signal to noise ratio. NCPHMs prepared by printing are favorably used for fluidic time delays. It is possible to tune the delay resulting from the NCPHM, by tuning the properties in the printed NC lines. The line width, number of lines, NC content in the lacquer and the chosen detergent can be influencing factors.

The NCPHMs can also be used for western blot applications. In this case they are preferably fully coated, e.g. by dip coating.

Another application of the NCPHMs of the present invention is their use as filter membranes. For this, cellulose paper sheets of suitable thickness and porosity can be coated with a nitrocellulose lacquer. As discussed above, the concentration of nitrocellulose in the lacquer can be used to define whether the pores of the paper shall be partially or completely filled with a porous nitrocellulose structure. By this method, the NCPHMs can be adjusted to the specific filtration conditions needed.

The entire disclosure of all applications, patents, and publications cited above and below as well as the corresponding application EP 20199154.4, filed Sep. 30, 2020, are hereby incorporated by reference. The present invention, without being limited thereby, is further illustrated by the following examples.

### EXAMPLES

### Used Chemicals and their Suppliers

### Nitrocellulose With the Following Properties:

| | | | D/a.u. | Nitrogen |
|---|---|---|---|---|
| Mn/g/mol | Mw/g/mol | Mz/g/mol | (D = Mw/Mn) | Content |
| 4.3 · 10⁴ | 1.0 · 10⁵ | 2.0 · 10⁵ | 2.4 | 11.8% |

Cellulose Acetate→Eastman CA-398-30 (G-01790B)
Aceton→Aceton AnalaR NORMAPUR Reag. Oph. Eur, Reag. USP, ACS (VWR 20066.330)
Ethanol→Ethanol (denatured with methylethylketone)>99.8% (Carl Roth, Art.-Nr. K928.3)
Butanol→1-Butanol, 99% (Alfa Aesar, L13171)
Isopropanol→2-Propanol>99.5% zur Synthese (Carl Roth, Art.-Nr. 9866.5, Lot)
Triton X®-100→Triton X®100 reinst (Carl Roth, Art.-Nr. 3051.3)
Glycerol→Glycerol BioXtra, >99% (GC), (Sigma Aldrich, G6279-1 L)

### Characterization Methods

### CFT Determination

Capillary Flow Time (CFT) is determined as follows: The NCPHMs are stored for 24 h under controlled climate conditions (23° C., 50% RH). To measure the CFT, samples are cut to 1.5 cm×4.0 cm strips. The fluid reservoir in the CFT stand is filled with 150 μL Milli-Q® water. The strip is placed in the reservoir and is leaning at an angle against the stand. A stopwatch is started when the strip comes into contact with the water and stopped when the flow front reaches the end of the strip (after 4 cm). The measured time is the CFT, given in seconds per 4 cm (s/4 cm).

### Coat Weight Determination

The coat weight of NCPHMs is described as the resulting mass of NC coating on the paper sheets after coating. This is determined via weighing a defined area of paper. In these experiments, disks with a diameter of 1.5 cm are punched out. The samples are stored under controlled climate conditions (23° C., 50% RH) for at least 24 h to equilibrate prior to the weighing. Knowing the mass of blank paper sheets (no NC) and the NCPHMs, the coat weight of NC can be determined by subtraction.

### Thickness Determination

The thickness of NCPHMs is determined via the RAINBOW thickness measurement system (Karl Schröder KG, Weinheim, Germany, fulfills DIN EN ISO 543, TAPPI T411 om-88 and SCAN P 7:75) or microscopic cross section images (SEM, CLSM).

### Example 1

The cotton linters fibers are from Buckeye Technologies (Cotton Linter Pulp, Grade 225 HSR-M, Lot. #: K45060636). Eucalypt sulfate pulp is from cmpc (Eucalyptus BHKP, G2056786). Paper sheets have been produced with grammages from 50 g/m²-155 g/m² (50 g/m², 75 g/m², 100 g/m² and 155 g/m²). Porosity of the papers was 60-70%. Paper thickness was 119 μm±3 μm for 50 g/m² paper and 177 μm±2 μm for 75 g/m² paper. Fiber length of cotton linters papers was 0.6 mm-0.9 mm and a fiber diameter from 16 μm-22 μm.

### General Fabrication of Paper Substrates for NCPHM Production

The cellulose pulp (dried fibers pressed as “cardboard”) is torn into small pieces (approx. 3 cm×3 cm) and soaked in tap water for 16 h. To separate the fibers from each other, max. 25 g of soaked pulp is transferred into a HAAGE/Estanit Disintegrator (type AG 04), filled with 2 L of tap water (fulfills: Merkblatt V/4/61 des Vereins der Zellstoff—und Papier-Chemiker und—Ingenieure and ISO 5263) and blend with a total of 75.000 turns. The resulting fiber suspension is transferred in a distribution container (fulfills: Vorschriften der Einheitsmethode gemäß Merkblatt V/6/61 des Vereins der Zellstoff—und Papier-Chemiker und—Ingenieure) and diluted to a resulting pulp concentration of 0.2%-1.0% (w/v).

The fiber suspension is stirred continuously, and every time pulp is taken to form a sheet, 3×1 liter of suspension is retrieved and added back to the container beforehand in order to remove agglomerations of cellulose at blind spots of the stirrer. For sheet forming a defined volume (depending on pulp concentration and desired grammage) of suspension is taken from the batch. The taken pulp is agitated continuously until sheet forming.

Sheet forming itself is done at the manual Haage/Estanit sheetformer BB (DIN EN ISO 5269-2 (DIN 54 358)) for lab standard papers. The taken fiber suspension is added to a total of 7 L freshwater and then mixed (with air) for 5 seconds. After calming for 5 seconds, the water is removed, and the resulting sheet is set to dry under vacuum at 93° C. in the drying unit. After minutes, the paper is accurately weighed (to determine the grammage) and further used for NCPHM fabrication.

### Example 2—Lacquer

The following table shows exemplary composition ranges for a suitable lacquer.

| component | Range of weight % |
|---|---|
| Nitrocellulose | 2.0-15 |
| Cellulose Esters | 0.0-1 |
| Acetone | 25-55 |
| one or more C1 to C4 alcohols | 10-65 |
| Glycerine | 0-1.5 |
| Detergent | 0-1 |
| Water (Milli-Q ®) | 5-25 |

The weight % of NC includes wet alcohol (NC with alcohol raw material).

### Example 3 Preparation of NCPHM

### Dip Coating

### Procedure:

The used cellulose papers are cut to 5.0 cm×10.0 cm dimensions and placed for 2 h in a climate cabinet (Binder SN 14-06929) with defined temperature and humidity to equilibrate.

For dip-coating the paper pieces are clamped on the horizontal and movable traverse of the dip-coater (description). Underneath the traverse, a beaker filled with NC lacquer is placed and the coating process is started.

The traverse travels down until the paper is completely immersed in the Lacquer. After resting for 5 seconds, the traverse travels up with the chosen coating speed as further defined below. For drying, the coated papers are left in the climate cabinet for 2 h.

To use the NCPHMs prepared with this method for CFT tests and in LFA applications, an additional detergent treatment is typically necessary.

### Varied Parameters/Ranges:

The chosen dip speeds for coating have been in a range of 80 mm/min-1000 mm/min (80, 120, 200, 300, 400, 500, 1000).

The NC content in the lacquer was 3.5%, 5% and 7% (w/w) respectively.

Temperature range was chosen between 18° C.-45° C. (18, 20, 25, 35, 45).

Humidity was varied between 18% RH-80% RH (18, 20, 25, 50, 75, 80).

### Characterization:

Total thickness of NCPHM after production is in the range from 136 μm-229 μm for hybrids prepared with 50 g/m² paper sheets and 183 μm-290 μm for hybrids prepared with 75 g/m² paper sheets.

The applied mass of NC on the paper (coat weight) was determined via gravimetric methods and is in the range of 4.2 g/m²-33.7 g/m² for hybrids prepared with 50 g/m² paper sheets and 5.6 g/m²-13.3 g/m² for hybrids prepared with 75 g/m². From this it follows that the NC amount in the total hybrid is 7.7%-40.3% (w/w) for 50 g/m² paper sheets and 6.9%-15.1% (w/w) for 75 g/m².

An important measured value is the capillary flow time (CFT) for later use as LFA membrane. CFT values are determined as described above. The CFT of the prepared NCPHMs was in a range from 128 s/4 cm-574 s/4 cm for hybrids prepared with 50 g/m² paper sheets and 148 s/4 cm-628 s/4 cm for hybrids prepared with 75 g/m² paper sheets. This range is broad due to many different variations made in different experiments. Within one batch of NCPHMs, the values are reproducible with a low margin of error. The most interesting combination of fabrication parameters (50 g/m² paper sheets, NC content 3.5% (w/w), 25° C., 75% RH, 500 mm/min dip speed) yielded CFT from 121 s/4 cm-133 s/4 cm.

### Blade Coating

Procedure: A blade coater (BYK Automatic Film Applicator type) is placed in a climate cabinet. The used cellulose papers are cut to 5.0 cm×10.0 cm dimensions and placed for 2 h in a climate cabinet with the defined temperature and humidity to equilibrate.

For blade-coating the paper pieces are clamped on a polymeric foil (PET, PP, PTFE) on the bed of the coater. A blade with a defined gap-width is put on the device on the paper substrate. 4 mL of NC lacquer are placed in front of the blade, and the coating process is started. The coating is performed by a controlled travel of the blade over the paper surface. After coating, the paper, together with the foil, is released from the clamp and placed in the climate cabinet for 2 h for drying.

To use the NCPHMs prepared with this method for CFT tests and in LFA applications, an additional detergent treatment is typically necessary.

### Varied Parameters/Ranges:

Coating speeds with this method have been chosen in a range of 50 mm/s-500 mm/s (in detail: 50, 100, 150, 200, 250, 400, 500). The gap size was in a range of 30 μm-90 μm (30, 60, 90).

The NC content in the lacquer was 3.5%, 5% and 7% (w/w) respectively. Temperature range was chosen between 18° C.-45° C. (18, 20, 25, 35, 45). Humidity was varied between 18% RH-80% RH (18, 20, 25, 50, 75, 80).

### Characterization

Total thickness of NCPHM prepared by this method is comparable to hybrids prepared by dip coating.

The coat weight of the prepared NCPHMs is in the range of 3.1 g/m²-4.5 g/m² for hybrids prepared with 50 g/m² paper sheets and 2.2 g/m²-8.4 g/m² for hybrids prepared with 75 g/m². From this it follows that the NC amount in the total hybrid is 5.8%-8.3% (w/w) for 50 g/m² paper sheets and 2.8%-10% (w/w) for 75 g/m².

The CFT of the prepared NCPHMs was in a range from 141 s/4 cm-721 s/4 cm for hybrids prepared with 50 g/m² paper sheets and 119 s/4 cm-481 s/4 cm for hybrids prepared with 75 g/m² paper sheets. The best combination of fabrication parameters (75 g/m² paper sheets, NC content 3.5% (w/w), 90 μm gap, 25° C., 75% RH, 200 mm/s casting speed) yielded CFT from 119 s/4 cm-129 s/4 cm.

### Size-Press Coating

### Procedure:

The temperature and humidity are determined with a hygrometer before the coating.

Cellulose paper is cut to 5.0 cm×10.0 cm dimensions. The coating speed and the linear pressure on the rollers are set. Afterwards, the NC lacquer (approximately 400 mL) is poured between the rollers (“immersion bath”). The paper sheets are manually inserted through the immersion bath between the rollers. After the sheets have passed the rollers, the sheets are taken with tweezers out of the press and dried for hanging at ambient temperature and humidity (determined before coating). To use the NCPHMs prepared with this method for CFT tests and in LFA applications, a detergent treatment is preferred.

### Varied Parameters/Ranges:

Coating speeds with this method have been chosen in a range of 1.0 m/min- 12 m/min (1, 3, 12). The line pressure (linear pressure) was in a range of 0.5 daN/cm-12.5 daN/cm (0.5, 6.5, 12.5).

The NC content in the lacquer was 3.5%.

Temperature was 23.5° C.

Humidity was 35.3% RH.

### Characterization:

Total thickness of NCPHM is almost the same compared to the used paper sheets.

The coat weight of the prepared NCPHMs is in the range of 0.60 g/m²-5.32 g/m². For the preparation, only cellulose papers with a grammage of 50 g/m² have been used. From this it follows that the NC amount in the total hybrid is 1.19%-9.62% (w/w).

The CFT of the prepared NCPHMs was in a range from 172 s/4 cm-390 s/4 cm. The best combination of fabrication parameters (50 g/m², NC content 3.5% (w/w), 23.5° C., 35.5% RH, 12.5 daN/cm, 3 m/min) yielded CFT of 172 s/4 cm.

### Line Printing

### Procedure:

Line printing is performed on a Hyrel System 30M 3D printer equipped with a syringe deposition system. For the application of the NC lacquer, a 500 μL Hamilton gastight syringe with a bent and flexible (45°) PP Luer-Lock-needle (20 G, 0.60 mm) is used. Alternatively, a dosing brush can be used instead of the needle. The paper sheets (5.0 cm×10.0 cm dimensions) are placed on a vacuum sinter-ceramics table to fix (avoid ripples) and align the sheets. During the printing process, the needle is in a slight contact with the paper. Printing speed is set to 600 mm/min with a dosing volume of 4 μL/cm resulting in approx. 3 mm broad lines. For this method, no climate control was applied to the printer. The printed lines are allowed to dry for 2 h at ambient temperature and humidity.

Depending on the later application of the material, one line (for protein barrier/depletion) or two lines (LFA membrane) at a distance of 10 mm are printed.

Also like the other NCPHMs, the printed lines typically need a detergent coating, otherwise the printed NC line acts as a hydrophobic barrier. The detergent treatment can be proceeded as described above, or by printing the detergent with 4 μL/cm and 600 mm/min directly on the dried NC line. Drying time after detergent printing was 2 h at ambient temperature and humidity.

### Varied Parameters/Ranges:

The grammage of the used cellulose papers was varied between 50 g/m²-155 g/m² (50, 75, 100, 155). NC content was chosen as 7% (w/w). Temperature and humidity were not controlled over the different experiments. Temperature was in a range of 20° C.-27° C., humidity was 35% RH-60% RH. Dosing volume was varied between 0.25 μL/cm-4.0 μL/cm (0.25, 0.5, 1.0, 2.0, 4.0). Only a dosing of 4 μL/cm leads to a complete penetration through the paper sheet.

### Characterization:

Thickness of the NCPHMs prepared via printing was not determined. Microscopy data leads to the conclusion that there is no change in thickness compared to the paper sheets.

Depending on the number of lines printed, the CFT ranges from 75 s/4 cm- 80 s/4 cm (1 line) and from 105 s/4 cm-125 s/4 cm (2 lines). Each printed line increases the CFT by about 20 s compared to the CFT of the used paper sheet (˜60 s/4 cm).

For coat weight calculation, it was assumed that a single test strip (2 lines printed with 4 μL/cm) has a dimension of 3.0 cm×0.5 cm. This results in 4 μL NC lacquer per test strip. To compare this, 666 test strips equals 1 m² of NCPHM. The calculated amount of needed NC is 0.13 g NC, which is 1.6 of a dip coated NCPHM (˜8 g/m²).

### Example 4 Pore Sizes/Particle Sizes/Porosity of Nitrocellulose Within the Hybrid Material

The following table shows characteristics of NCPHMs according to the present invention.

| | | | Average | Average |
|---|---|---|---|---|
| | Type of | | Particle | Pore |
| | paper | Porosity/% | Size/μm | Size/μm |
| NC Paper | L50-L7M- | 78.3 | 0.640 | 1.448 |
| Hybrid | 120.25.50 | | | |
| NC Paper | L7-75-0E top | 86.4 | 0.515 | 1.544 |
| Hybrid | layer | | | |
| NC Paper | L7-75-0E | 84.1 | 1.177 | 3.224 |
| Hybrid | cross | | | |
| | section | | | |

### Example 5 Lateral Flow

NCPHMs are tested for application in a classical lateral flow test setup using hCG (human chorionic gonadotropin) pregnancy immunoassay and HBsAg (Hepatitis B surface antigen) immunoassay as model systems. The NCPHMs are compared to classical nitrocellulose membranes and paper references (blank base paper without NC).

Test line solutions containing 1 mg/ml anti-hCG or anti-HBsAg specific capture antibodies in 10 mM MES (pH 6.5) and control line solution containing 1 mg/ml goat anti-mouse capture antiserum (M8890, Merck) are striped onto NCPHMs or reference substrates using a HYREL 3D System 30M printing device (Hyrel 3D) (dispensed volume: 0.1 μl/mm, speed: 1 cm/s) to yield 0.5 μg capture protein per strip. Substrates are dried 2 h at 37° C.

Anti-hCG or anti-HBsAg gold conjugates (OD 2) in 5% (w/v) trehalose are applied onto glass fiber conjugate pads (GFDX203000, Merck) and dried overnight at ambient temperature.

Additionally, 2 mL of anti-hCG (10-C25E, Fitzgerald) conjugated to 400 nm latex beads (K1-030 bleu, Lot: M2481/35, Estapor) in conjugate buffer (microsphere concentration 0.065% w/v) is applied to glass fiber pads (GFDX203000, Merck) to prepare latex-conjugate pads. The soaked glass fiber pads are dried overnight at ambient temperature, followed by drying for 2 h at 37° C. on a wire tray.

NCPHMs and references (0.5 cm×3 cm) are assembled together with conjugate pads (0.5 cm×0.5 cm), cellulose sample pads (0.5 cm×2.5 cm) (CFSP223000, Merck; treated with 10 mM Tris-HCl pH 8.2, 1% (w/v) Tween 20, 0.75% (w/v) BSA) and absorbent pads (0.5 cm×2.5 cm) (CFSP223000, Merck) into a classical lateral flow test setup (FIG. 1).

NCPHM functionality in lateral flow tests was analyzed by either hCG or HBsAg concentration series spiked in respective buffer matrices, using synthetic urine (S-020, Cerilliant) for hCG LFT and human serum/PBS 1:1 diluent (H4522, Sigma) for HBsAg LFT.

### One-Piece Lateral Flow Test setup

In additional to the classical LFT setup, NCPHMs are tested in a new “one-piece” approach using hCG (human chorionic gonadotropin) pregnancy immunoassay and HBsAg (Hepatitis B surface antigen) immunoassay as model systems.

One-piece LFT approach is based on a single paper (e.g. cotton linters) substrate with functionalized LFT areas (FIG. 2):
Sample area (start zone)
   0.5 cm×2.5 cm sample area of one-piece LFTs were soaked with 20 μl 10× sample pad buffer (100 mM Tris-HCl pH 8.2, 10% (w/v) Tween 20, 7.5% (w/v) BSA).
Conjugate area
   0.5 cm×0.5 cm conjugate area soaked with 20 μl of respective antibody-gold conjugate (OD 2 in 5% trehalose, 20% sucrose, 1% BSA) by pipetting in steps of 5-times 4 μl and placing on a 40° C. heat plate for in-between drying
Test and control antibody area (detection zone)
   Printed nitrocellulose lines on paper (e.g. Cotton linters, Eucalyptus) according to NCPHM production by line printing
Absorbent area
   0.5 cm×2.5 cm untreated absorbent area

Application of antibody test and control line is performed according to classical LFT setup. Test line solutions containing 1 mg/ml anti-hCG or anti-HBsAg specific capture antibodies in 10 mM MES (pH 6.5) and control line solution containing 1 mg/ml goat anti-mouse capture antiserum (M8890, Merck) are striped onto NC/paper hybrid lines of one-piece LFT using a HYREL 3D System 30M printing device (Hyrel 3D) (dispensed volume: 0.1 μl/mm, speed: 1 cm/s) to yield 0.5 μg capture protein per strip. Substrates are dried 2 h at 37° C.

One-piece LFT functionality was analyzed by either hCG or HBsAg concentration series spiked in respective buffer matrices, using synthetic urine (S-020, Cerilliant) for hCG LFT and human serum/PBS 1:1 diluent (H4522, Sigma) for HBsAg LFT.

FIG. 2 shows the scheme of a “one-piece” lateral flow assay obtained by line printing of NC lacquer prior to antibody application on test- and control line.

### Application Tests

Classical LFT setups have been prepared as described above with NCPHMs as LFT membranes.

The hCG lateral flow assays are performed with anti-hCG gold conjugates (40 nm gold beads) and anti-hCG latex conjugates (400 nm latex beads) and compared to state of the art LFAs with commonly used NC membranes and pure paper sheets.

Compared to the NC membrane (standard membrane for LFAs), the dip-coated and printed NCPHMs are also able to detect the lowest hCG concentration of 25 mIU/mL. The widths of test and control lines are greater on NCPHMs compared to the NC membrane. The blank paper control sample shows very broad lines. The lowest concentration of 25 mIU/mL is hard to see. In principle, the LFA can be performed on blank papers, but the signal intensity is bad. This indicates a necessity to apply NC to get better signal intensity.

The one-piece LFT setup has been prepared as described above followed by detergent coating. The antibodies are applied after the detergent coating on test and control line. LFT was made with anti-hCG gold conjugates. The NCPHM was fabricated by line printing as described above on 75 g/m² paper sheets. The one-piece LFT only consists of a paper strip, with two printed NC lines of each 4 μL/cm (for test- and control line), printed antibodies on test and control lines, a conjugate area (anti-hCG conjugate pipetted on the strip) and the sample area. No assembly of different functional parts (e.g. conjugate pad) was performed.

The capillary flow times of the NCHPMs ranged from 105 s/4 cm-125 s/4 cm, and the calculated NC mass per test strip was 0.2 mg.

The anti-hCG lateral flow assays were compared to state of the art LFAs with commonly used NC membranes.

The one-piece LFT showed comparable sensitivity in the anti-hCG pregnancy assay compared to the classical LFT setup on a NC membrane. The lowest concentration of hCG (25 mIU/mL) was detectable. The intensity of test and control line is similar to the reference, but the lines are slightly wider.

### Example 6 Protein Depletion

The NCHPM was fabricated by line printing on 50 g/m² paper sheets with only one NC line applied at 4 μL/cm. The NCPHMs are placed for 10 min in a tray filled with detergent solution at room temperature (19° C.-25° C.). Afterwards, the NCPHMs are taken out and dried at room temperature overnight.

The strip for protein depletion was cut into 0.5 cm×5 cm dimensions. The applied amount of NC was calculated to be 100 μg per strip.

For the protein depletion experiment, FITC-labelled BSA was diluted in PBS buffer (pH 7.4) to a protein concentration of 2.5 ng/μL. A FITC-dextran solution with an identical concentration is prepared in PBS (pH 7.4) as reference.

The NCPHM strip (0.5 cm×8.0 cm) is placed on PMMA microscope slides, with 3 mm-5 mm overhang on the edge of a lab-jack. The end of the strip is placed on a small heating foil (40° C.). To start the fluid flow, a PBS-buffer filled petri dish is lifted with a second lab-jack so that the paper strip touches the surface of the water. After 1.5 cm flow distance, 5 μL of protein (dextran as reference) solution is applied on the flow front (wet out flow). This corresponds to a total mass of 12.5 ng of protein/dextran. After the sample is applied, the flow is maintained for 15 minutes. Afterwards, the strips are dried at ambient temperature prior to microscopic analysis and characterization. A scheme of the experimental set up is shown in FIG. 4.

The microscopic evaluation of the experiment shows that all BSA (12.5 ng) is captured by the applied NC line (100 μg). At the end of the strip, no BSA is detectable. The reference experiment shows that almost all dextran has passed the NC line despite its higher mass.

### Example 7

Printed lines of NCPHMs have shown delaying and barrier properties on fluid flow through the material. The hybrids have been prepared by line printing (see above) and treated with detergents by complete immersion. Two different detergents have been used (SDS, SDBS). FIG. 5 shows the scheme of prepared hybrids and capillary flow times of hybrids without detergent and detergents.

Two different layouts have been used (FIG. 5, left). The graph (FIG. 5, right) shows the CFT of blank paper (˜60 s) and hybrids with one and two printed lines with and without detergent. The samples without detergent stop at the first printed line after 8-10 s. The fluid does not pass the line, even after 1 h. Samples prepared with one line (light grey bars) have a CFT around ˜80 s independent from the chosen detergent. This is 20 s more than the blank linters (black) and is equal to a slower fluid flow speed. Samples with two printed lines (dark grey bars) show another delay. The CFT is between 110-120 s, whereas the SDBS samples show a lower CFT compared to SDS samples.

1. A nitrocellulose coated paper (NCPHM) comprising a cellulose paper sheet partially or fully coated with nitrocellulose and a detergent.

2. A nitrocellulose coated paper (NCPHM) according to claim 1 which is prepared by
a) fully or partially contacting a cellulose paper sheet with a lacquer comprising nitrocellulose in a solvent in which nitrocellulose is soluble and a nonsolvent pore former which is a solvent in which nitrocellulose is insoluble,
b) drying the coated paper sheet and
c) fully or partially treating the dried paper sheet of step b) with a detergent solution.

3. A nitrocellulose coated paper (NCPHM) according to claim 1 characterized in that the NCPHM has a thickness between 100 and 300 μm.

4. A nitrocellulose coated paper (NCPHM) according to claim 1, characterized in that the cellulose paper sheet has pores with sizes between 3 μm and 30 μm.

5. A nitrocellulose coated paper (NCPHM) according to claim 1, characterized in that the nitrocellulose coating has pores between 0.2 μm and 15 μm.

6. A nitrocellulose coated paper (NCPHM) according to claim 1, characterized in that the nitrocellulose coated paper comprises between 1% and 40% nitrocellulose.

7. A nitrocellulose coated paper (NCPHM) according to claim 1, characterized in that the lacquer comprises 1 to 20% (w/w) of nitrocellulose.

8. A nitrocellulose coated paper (NCPHM) according to claim 1, characterized in that the lacquer comprises 0.05 to 1% (w/w) of a detergent.

9. A nitrocellulose coated paper (NCPHM) according to claim 1, characterized in that the detergent of step c) is an anionic detergent.

10. A method for preparing nitrocellulose coated paper (NCPHM) by
a) fully or partially contacting a cellulose paper sheet with a lacquer comprising nitrocellulose in a solvent in which nitrocellulose is soluble and a nonsolvent pore former
b) drying the coated paper sheet
c) fully or partially treating the dried paper sheet of step b) with a detergent solution.

11. Method according to claim 10, characterized in that in step a) the cellulose based paper is contacted with the lacquer by dip coating, blade coating, size press coating or printing.

12. A lateral flow device comprising a nitrocellulose coated paper according to claim 1.

13. A lateral flow device according to claim 12, characterized in that the lateral flow device comprises a single cellulose paper sheet incorporating at least a start zone and a detection zone which is only partly coated with nitrocellulose and a detergent.

14. A lateral flow device according to claim 12 characterized in that it comprises a nitrocellulose coated area for protein depletion between the start zone and the detection zone.

15. A lateral flow test, filtration device, protein barrier, or western blot assay comprising a nitrocellulose coated paper, wherein the nitrocellulose paper is one according to claim 1.
